(19) United States
(12) Patent Application Publication
(10) Pub. No.: US 2022/0409813 A1
(43) Pub. Date: Dec. 29, 2022

(54) PRESSURE-REGULATING CONNECTOR FOR INFUSION

(71) Applicant: CAREFUSION 303, INC, San Diego, CA (US)

(72) Inventor: Janice R. PAK, Centennial, CO (US)

(21) Appl. No.: 17/852,027

(22) Filed: Jun. 28, 2022

Related U.S. Application Data
(60) Provisional application No. 63/216,350, filed on Jun. 29, 2021.

(51) Int. Cl. A61M 5/168; A61M 5/145

(52) U.S. Cl. CPC A61M 5/16881; A61M 5/14586; A61M 5/16813; A61M 2039/229

(57) ABSTRACT

A system for controlled delivery of a medical fluid may include an elastomeric pump, a valve member fluidly coupled to the elastomeric pump at one side and a catheter at an opposite side for delivering the medical fluid to a patient, and tubing fluidly coupling the elastomeric pump and the valve member. The elastomeric pump may include an elastomeric membrane and a shell surrounding the elastomeric membrane. The pressure of the medical fluid may be greater than a pressure of the vein to keep the vein open. The valve member may be movable between an initial closed position, a fully open position where the medical fluid is delivered to the vein of the patient at a predetermined fluid flow rate, and at least one intermediate open flow position where the medical fluid is delivered to the vein of the patient at a flow rate below the predetermined fluid flow rate.

### CROSS-REFERENCE TO RELATED APPLICATIONS

This application claims the benefit of U.S. Provisional Application No. 63/216,350, filed Jun. 29, 2021, the entirety of which is incorporated herein by reference.

### TECHNICAL FIELD

The present disclosure generally relates to administration of medical fluids to a patient by means of an elastomeric pump, and particularly to improved systems and devices for controlled delivery of a medical fluid, including an elastomeric pump and a keep-vein-open vascular line assist device.

### BACKGROUND

Catheters are commonly used for a variety of infusion therapies. For example, catheters may be used for infusing fluids, such as saline solution, various medicaments, and total parenteral nutrition, into a patient. Catheters may also be used for withdrawing blood from the patient.

A common type of catheter is an over-the-needle peripheral intravenous (“IV”) catheter. As its name implies, the over-the-needle catheter may be mounted over an introducer needle having a sharp distal tip. A catheter assembly may include a catheter hub, the catheter extending distally from the catheter hub, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

The description provided in the background section should not be assumed to be prior art merely because it is mentioned in or associated with the background section. The background section may include information that describes one or more aspects of the subject technology.

### SUMMARY

In accordance with various embodiments of the present disclosure, a system for controlled delivery of a medical fluid may include an elastomeric pump containing the medical fluid, a valve member fluidly coupled to the elastomeric pump at one side and a catheter at an opposite side for selectively delivering the medical fluid to a vein of a patient, and a tubing interposed between and fluidly coupling the elastomeric pump and the valve member. The elastomeric pump may include an elastomeric membrane defining a chamber containing the medical fluid, and a shell surrounding the elastomeric membrane. A pressure of the medical fluid may be greater than a pressure of the vein by a predetermined value to keep the vein open. The valve member may be movable between an initial closed position, a fully open position where the medical fluid is delivered to the vein of the patient at a predetermined fluid flow rate, and at least one intermediate open flow position where the medical fluid is delivered to the vein of the patient at a flow rate below the predetermined fluid flow rate.

In accordance with various embodiments of the present disclosure, a system for controlled delivery of a medical fluid may include an elastomeric pump containing the medical fluid at a first pressure, a valve member fluidly coupling the elastomeric pump and a catheter, and a tubing interposed between and fluidly coupling the elastomeric pump, the valve member, and the catheter. The elastomeric pump may include an elastomeric membrane defining a chamber containing the medical fluid, and a shell surrounding the elastomeric membrane. The valve member may include (i) a base portion including an inner lumen, an inlet aperture, and an outlet aperture, and (ii) a body portion including a stem disposed at least partially within the inner lumen and including a handle. The stem may have a plurality of flow channels extending therethrough and defining fluid pathways of different sizes. The valve member may fluidly couple the elastomeric pump and the catheter via each of the flow channels for selectively delivering the medical fluid to a patient's vein having a second pressure less than the first pressure by a predetermined value to keep the patient's vein open.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The following figures are included to illustrate certain aspects of the embodiments and should not be viewed as exclusive embodiments. The subject matter disclosed is capable of considerable modifications, alterations, combinations, and equivalents in form and function, as will occur to those skilled in the art and having the benefit of this disclosure.

FIG. 1A illustrates a system for controlled delivery of a medical fluid including an elastomeric pump and a keep-vein-open vascular line assist device, in accordance with some embodiments of the present disclosure.

FIG. 1B illustrates a cross-sectional view of the keep-vein-open vascular line assist device, in accordance with some embodiments of the present disclosure.

FIG. 1C illustrates an exploded cross-sectional view of the keep-vein-open vascular line assist device, in accordance with some embodiments of the present disclosure.

FIG. 2 is an isometric view of the keep-vein-open vascular line assist device in an initial closed condition, in accordance with some embodiments of the present disclosure.

FIG. 3 is an isometric view of the keep-vein-open vascular line assist device in a first flow condition, in accordance with some embodiments of the present disclosure.

FIG. 4 is an isometric view of the keep-vein-open vascular line assist device in a second flow condition, in accordance with some embodiments of the present disclosure.

FIG. 5 is an isometric view of the keep-vein-open vascular line assist device in a third flow condition, in accordance with some embodiments of the present disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided for certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

It is often necessary to intravenously supply a patient with medical fluids at a controlled rate over a long period of time in order to keep the patient's vein open after a catheter has been placed in the vein of the patient. Currently, manual syringe flushes are administered in order to keep veins open after a catheter has been placed. However, this is often an arduous task for clinicians and does not always result in keeping the vein open as there isn't a continuous flushing of the vascular access line. How frequently flushes are administered also depends on the patient, and out-patients may not get regular flushes resulting in potential occlusions and infections.

Various embodiments of the present disclosure are directed to providing systems and devices for controlled delivery of a medical fluid. The systems and devices for controlled delivery of a medical fluid may include an elastomeric pump and a keep-vein-open vascular line assist device. The keep-vein-open vascular line assist device (also referred to herein as a valve member, check valve, or stop cock valve) is advantageously capable of keeping a vascular access line open when not in continuous use. The keep-vein-open vascular line assist device of the various embodiments described herein may advantageously provide continuous flushing of the vascular access line under controlled pressure until the elastomeric pump is emptied. Accordingly, the potential harm to patients associated with catheter occlusions and catheter infections may be reduced or avoided. The keep-vein-open vascular line assist device may also help increase compliance to vascular access maintenance regulations and reduce the amount of time spent by medical professionals manually flushing the line with a syringe. Further advantageously, the keep-vein-open vascular line assist device may also provide a controlled amount of fluid to the patient—especially for those who may be more sensitive to fluid build-up.

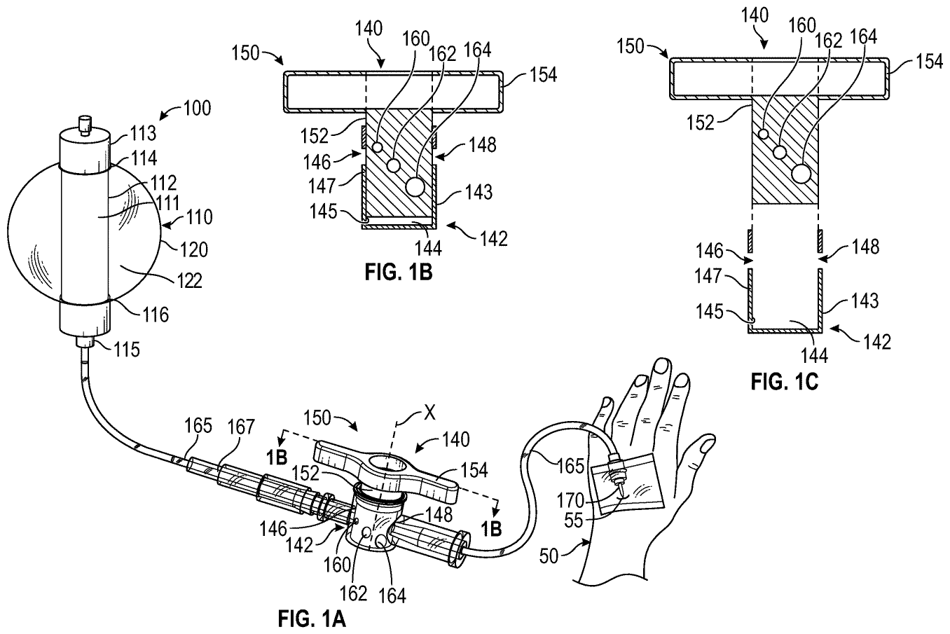

FIG. 1A illustrates a system for controlled delivery of a medical fluid including an elastomeric pump and a keep-vein-open vascular line assist device, in accordance with some embodiments of the present disclosure. According to various embodiments of the present disclosure, a system 100 for controlled delivery of a medical fluid may include an elastomeric pump 110 and a keep-vein-open vascular line assist device 140 (also referred to herein as a valve member 140) fluidly coupled to the elastomeric pump 110. As depicted, the valve member 140 may be coupled to the elastomeric pump 110 at one side via the tubing 165 and in some instances a luer connector. The valve member 140 may further be coupled to a catheter 170 at an opposite side for selectively delivering the medical fluid to a vein 55 of a patient 50.

According to various embodiments of the present disclosure, the elastomeric pump 110 for use with the system 100 for controlled delivery of a medical fluid includes an elastomeric membrane 112 which is expandable and contractible and which defines a chamber 111 therein for receiving and containing a volume of a medical fluid such as a medical liquid, gas, oxygen, or a combination of a liquid and a gas. In operation, the chamber 111 may exert pressure on the medical fluid contained therein to force the medical fluid from the elastomeric membrane 112 in order to dispense the medical fluid through the catheter 170 to the patient undergoing treatment.

In some embodiments, the elastomeric membrane may further include an inlet port 114 for connecting to a syringe or similar device for filling the elastomeric pump 110. In some embodiments, the elastomeric membrane 112 has formed therein an outlet port 116 that is fluidly communicated with the chamber 111 and the valve member 140 for connecting to the catheter 170 for dispensing the medical fluid from the elastomeric pump.

According to various embodiments of the present disclosure, the elastomeric pump 110 may include a shell 120 which defines an interior space 122 in which the elastomeric membrane 112 is situated. The IV tubing 165 which may be in the form of a flexible, medical grade tubing, may be coupled to the elastomeric membrane 112 and the valve member 140 and may define a fluid passageway or lumen 167 which is in communication with the valve member 140 and the outlet port 116 and chamber 111 of the elastomeric membrane 112.

According to various embodiments of the present disclosure, the expandable elastomeric membrane 112 may be in the form of a cylindrical tube having a first open axial end corresponding to the inlet port 114, and a second open axial end corresponding to the outlet port 116, opposite the first open axial end. In some embodiments, the elastomeric membrane 112 may be formed of a medical grade, low durometer, silicone material. In some embodiments, silicone may be used as the material for its elastomeric properties. Although the elastomeric membrane 112 may be preferably formed from a silicone material, other elastomers and materials may be used, such as a polymer or synthetic rubber or a latex material.

FIG. 1B illustrates a cross-sectional view of the keep-vein-open vascular line assist device (also referred to herein as the valve member 140), in accordance with some embodiments of the present disclosure. FIG. 1C illustrates an exploded cross-sectional view of the keep-vein-open vascular line assist device (also referred to herein as the valve member 140), in accordance with some embodiments of the present disclosure. In some embodiments, the valve member 140 may be in the form of a check valve. In some embodiments, as illustrated in FIGS. 1A-1C, the valve member 140 may be a stopcock valve. For example, according to various embodiments of the present disclosure, the stopcock valve 140 may include a base portion 142 including an inner lumen 144, an inlet aperture 146, and an outlet aperture 148.

As depicted, the base portion 142 may be in the form of a cylindrically extending longitudinal body having outer surface 143, an inner surface 145 defining the lumen 144, and a sidewall 147 defined between the inner surface 145 and the outer surface 143. In some embodiments, the cylindrically extending longitudinal body may include an aperture extending longitudinally through opposing sides of the sidewall 147 of the base portion 142 for fluidly coupling the base portion 142 to the lumen 167 of the IV tubing 165. For example, in some embodiments, an aperture extending longitudinally through opposing sides of the sidewall 147 of the base portion 142 may include an inlet aperture 146 coupled to a first segment of the IV tubing 165 and an outlet aperture coupled to a second segment of the IV tubing 165. The inlet and outlet aperture 146 and 148 may be axially aligned at positions mirroring each other on the cylindrically extending longitudinal body of the base portion 142.

According to various embodiments of the present disclosure, the valve member 140 may further include a body portion 150 including a stem 152 disposed at least partially within the lumen 144 and a handle 154. As depicted, the stem 152 may be in the form of a body including at least one flow channel extending laterally though the body. For example, in some embodiments, the valve stem 152 may include a plurality of flow channels extending laterally though the body. In particular, the stem 152 may include a plurality of flow channels 160, 162, and 164 extending through the stem 152. Each flow channel 160, 162, 164, when aligned with the inlet and outlet apertures 146 and 148 of the base portion 142, may define a fluid pathway through the valve member 140. In the illustrated embodiments, the stem 152 includes the first flow channel 160, the second flow channel 162, and the third flow channel 164, however the various embodiments of the present disclosure are not limited to the aforementioned configuration. In some embodiments, the stem 152 may include more than flow channels, and in other embodiments, the stem 152 may include two or less flow channels.

In some embodiments, the flow channels 160, 162, 164 may have different sizes thereby defining fluid flow passages of different sizes though the valve member 140. For example, in some embodiments, the first flow channel 160 may have a first diameter, the second flow channel 162 may have a second diameter, and the third flow channel may have a third diameter. In some embodiments, the sizes or diameters of the flow channels 160, 162, 164 may be different. For example, the second diameter may be larger than the first diameter, and the third diameter may be larger than the second diameter. Although the flow channels 160, 162, and 164 are described herein with the size or diameter of the second flow channel 162 being larger than the size or diameter of the first flow channel 160 and the size or diameter of the third flow channel being larger than the size or diameter of second flow channel, the various embodiments of the present disclosure are not limited to the aforementioned configuration. In some embodiments, the order may be reversed with the first flow channel 160 being larger than the second flow channel 162 and the second flow channel 162 being larger than the third flow channel 164.

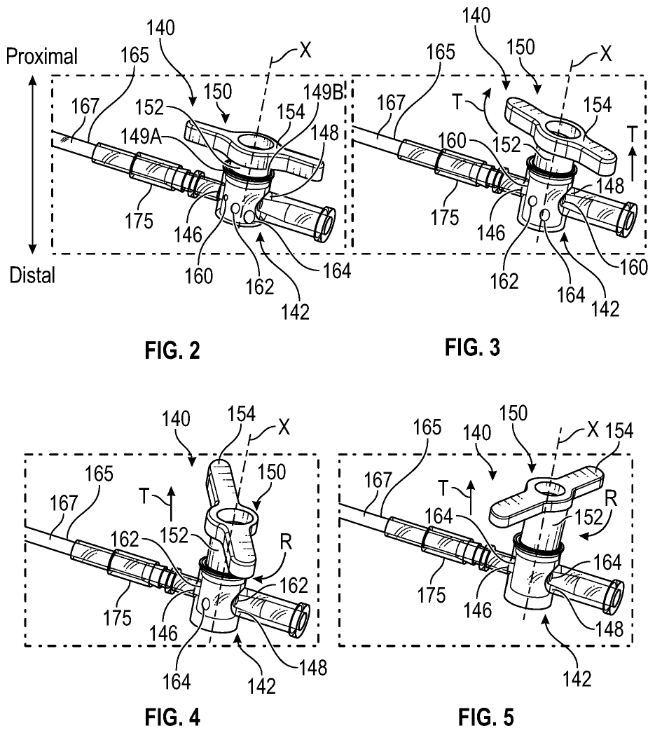

As shall be described in further detail with respect to FIGS. 2-5, the first, second and third flow channels 160, 162, and 164 having different diameters may allow for different flow rates of the medical fluid flowing through the valve member to the patient's vein 55. For example, the first flow channel having a smallest diameter may allow for a slower fluid flow rate than the second flow channel, the second flow channel 162 may in turn allow for a smaller flow rate than the third flow channel 164, but a larger flow rate than the first flow channel 160, and the third flow channel may allow for the largest flow rate of the three flow channels 160, 162 and 164.

The aforementioned configuration with the plurality of flow channels having different sizes may advantageously allow the valve member 140 to be rotatable between (i) an initial closed position where none of the flow channels 160, 162, and 164 are aligned with the inlet and outlet apertures 146 and 148, (ii) a fully open position where the third flow channel 164 is aligned with the inlet and outlet apertures 146 and 148 and the tubing lumen 167, and the medical fluid is delivered to the vein of the patient at a first (largest) fluid flow rate and/or pressure, and (iii) intermediate flow positions where the first flow channel 160 is aligned with the inlet and outlet apertures 146 and 148 and the tubing lumen 167, and the second flow channel 162 is aligned with the inlet and outlet apertures 146 and 148 and the tubing lumen 167 such that the medical fluid is delivered to the vein of the patient at second and third flow rates, respectively which are lower than the first fluid flow rate to keep the vein 55 open.

FIG. 2 is an isometric view of the keep-vein-open vascular line assist device in an initial closed condition, in accordance with some embodiments of the present disclosure. According to various embodiments of the present disclosure, the plurality of flow channels 160, 162, and 164 may be longitudinally spaced apart from each other along a circumference of the stem 152. For example, in some embodiments, each of the flow channels 160, 162, and 164 may be positioned at different heights along the length of the stem 152.

In some embodiments, the inner surface 145 of the base portion 142 may include a plurality of threads 149A and the stem 152 may also include a plurality of complimentary threads 149B on an outer surface thereof. The complimentary threads 149A and 149B may be designed such that they may engage each other to rotatably couple the base portion 142 and the body portion 150 of the valve member 140. For example, in an engaged configuration of the complimentary threads 149A and 149B, the body portion 150 may be rotatable about a central axis X of the base portion 142 to translate the body portion 150 proximally and distally within the lumen 144 of the base portion 142.

According to various embodiments of the present disclosure, the valve member 140 may define several states or flow conditions of the keep-vein-open vascular line assist device. FIG. 2 depicts an initial closed or no flow condition of the keep-vein-open vascular line assist device. In the closed or no flow condition none of the first, second or third flow channels 160, 162, and 164 are aligned with the inlet and outlet apertures 146 and 148 (also referred to herein as the aperture pair) of base portion 142. Accordingly, the stem 152 occludes the inner lumen 144 of the base portion 142 and blocks fluid communication between the elastomeric pump 110 and the catheter 170. For example, the stem 152 may block fluid communication between the first segment of the IV tubing 165 at the inlet aperture 146 and the second segment of the IV tubing 165 at the outlet aperture 148 such that the medical fluid (e.g., IV fluid) is not delivered to the catheter 170 and the patient.

FIG. 3 is an isometric view of the keep-vein-open vascular line assist device in a first flow condition, in accordance with some embodiments of the present disclosure. As depicted in FIG. 3, in the first flow condition, the first flow channel 160 may be co-axially aligned with the inlet and outlet apertures 146 and 148 of the base portion 142 and the lumen 167 of the tubing 165. Accordingly, the stem 152 fluidly communicates the elastomeric pump 110 and the catheter 170 via the first flow channel 160. For example, in the first flow condition the first flow channel 160 may fluidly communicate the first segment of the IV tubing 165 at the inlet aperture 146 and the second segment of the IV tubing 165 at the outlet aperture 148 such that the medical fluid (e.g., IV fluid) may be delivered to the catheter 170 and the patient at a first predetermined fluid flow rate which may be based in part on the size or diameter of the first flow channel. Since the size or diameter of the first flow channel 160 may be smaller than that of the second and third flow channels 162 and 164, the first predetermined fluid flow rate may in turn be lower than the fluid flow rates through the second and third flow channels 162 and 164.

The aforementioned configuration may be advantageous in that the valve member 140 may be rotated to the first flow condition when it is desired that a reduced or minimal flow of the medical fluid be administered to the patient for the purpose of keeping the patient's vein open. For example. As depicted in FIG. 3, the handle 154 of the valve member 140 may be rotated clockwise and the body portion 150 translated upwards or proximally to align the first flow channel 160 with the inlet and outlet apertures 146 and 148 of the base portion and the lumen 167 of the tubing 165. Accordingly, the first flow condition may deliver the medical fluid to the patient at a decreased flow rate as compared to the second and third flow conditions described below with respect to FIGS. 4 and 5.

FIG. 4 is an isometric view of the keep-vein-open vascular line assist device in a second flow condition, in accordance with some embodiments of the present disclosure. As depicted in FIG. 4, in the second flow condition, the second flow channel 162 may be co-axially aligned with the inlet and outlet apertures 146 and 148 of the base portion 142 and the lumen 167 of the tubing 165. Accordingly, the stem 152 fluidly communicates the elastomeric pump 110 and the catheter 170 via the second flow channel 160. For example, in the second flow condition the second flow channel 162 may fluidly communicate the first segment of the IV tubing 165 at the inlet aperture 146 and the second segment of the IV tubing 165 at the outlet aperture 148 such that the medical fluid (e.g., IV fluid) may be delivered to the catheter 170 and the patient at a second predetermined fluid flow rate which may be based in part on the size or diameter of the second flow channel 162. Since the size or diameter of the second flow channel 162 may be larger than that of the first flow channel 160 but smaller than that of the third flow channel 164, the second predetermined fluid flow rate may in turn be greater than the first predetermined fluid flow rate, but lower than the fluid flow rate through the third flow channel 164.

The aforementioned configuration may be advantageous in that the valve member 140 may be rotated to the second flow condition when it is desired that a reduced or minimal flow of the medical fluid be administered to the patient for the purpose of keeping the patient's vein open. For example, as depicted in FIG. 3, the handle 154 of the valve member 140 may be rotated further clockwise and the body portion 150 translated further upwards or proximally to align the first flow channel 160 with the inlet and outlet apertures 146 and 148 of the base portion and the lumen 167 of the tubing 165. Accordingly, the first flow condition may deliver the medical fluid to the patient at an intermediate flow rate which is greater than the first predetermined fluid flow rate in the first flow condition described above, but lower than the third flow condition described below. The second flow condition therefore may advantageously be activated to deliver medical fluid to patients with sensitive veins or veins otherwise not capable of handling flow rates or pressures of medical fluid on the magnitude of the third flow condition described below with respect to FIG. 5.

FIG. 5 is an isometric view of the keep-vein-open vascular line assist device in a third flow condition, in accordance with some embodiments of the present disclosure. As depicted in FIG. 5, in the third flow condition, the third flow channel 164 may be co-axially aligned with the inlet and outlet apertures 146 and 148 of the base portion 142 and the lumen 167 of the tubing 165. Accordingly, the stem 152 fluidly communicates the elastomeric pump 110 and the catheter 170 via the third flow channel 164. For example, in the third flow condition the third flow channel 164 may fluidly communicate the first segment of the IV tubing 165 at the inlet aperture 146 and the second segment of the IV tubing 165 at the outlet aperture 148 such that the medical fluid (e.g., IV fluid) may be delivered to the catheter 170 and the patient at a first predetermined fluid flow rate which may be based in part on the size or diameter of the third flow channel 164. Since the size or diameter of the third flow channel 164 may be larger than that of the first and second flow channels 160 and 162, the third predetermined fluid flow rate may in turn be greater than the fluid flow rates through the first and second flow channels 160 and 162.

The aforementioned configuration may be advantageous in that the valve member 140 may be rotated to the third flow condition when it is desired that maximum flow of the medical fluid be administered to the patient, for example during the normal course of administering the medical fluid. Accordingly, the third flow condition may be a fully open position of the valve member 140. For example, as depicted in FIG. 5, the handle 154 of the valve member 140 may be rotated further clockwise and the body portion 150 translated further upwards or proximally to align the third flow channel 164 with the inlet and outlet apertures 146 and 148 of the base portion 142 and the lumen 167 of the tubing 165. Accordingly, the third flow condition may deliver the medical fluid to the patient at an increased flow rate as compared to the second and third flow conditions described above with respect to FIGS. 3 and 4.

According to various embodiments of the present disclosure, the elastomeric pump 110 uses pressure of the medical fluid contained in the elastomeric membrane to infuse the medical fluid into the IV tubing 165. The elastomeric pump may be pre-filled with saline/sterile fluid to a set pressure that exceeds vascular pressure within safety limits, without causing risk of damage/injury to the patient's vein. When the elastomeric pump 110 gets filled, the elastomeric membrane stretches under the medical fluid pressure. As the elastomeric membrane 112 continues to stretch, the chamber 111 of the elastomeric membrane 112 may exert pressure on the medical fluid contained therein to force the medical fluid from the elastomeric membrane 112 and drive the medical fluid through the IV tubing 165. In some embodiments, the IV tubing 165 may be primed before connecting to the catheter 170 and associated patient vascular line. After connecting to the patient vascular line, the valve member may be rotated to the either of the first, second, or third flow conditions in order to dispense the medical fluid and to safely keep the vascular line open. During administration of the medical fluid to the patient in either the first, second, or third flow conditions, the pressure of the medical fluid in the elastomeric pump 110 advantageously stays above the threshold of vascular pressure to keep the patient's vein 55 open and clear.

In operation, when full flow of the medical fluid is desired (e.g., the third flow condition), the body portion 150 of valve member 140 may be rotated such that the third flow channel 164 may be co-axially aligned with the inlet and outlet apertures 146 and 148 of the base portion 142 and the lumen 167 of the tubing 165. Accordingly, the stem 152 may fluidly communicate the elastomeric pump 110 and the catheter 170 via the third flow channel 164 in order to dispense the medical fluid to the patient.

According to various embodiments, when reduced flow of the medical fluid is desired (e.g., the second flow condition), the body portion 150 of valve member 140 may be rotated such that the second flow channel 162 may be co-axially aligned with the inlet and outlet apertures 146 and 148 of the base portion 142 and the lumen 167 of the tubing 165. Accordingly, the stem 152 may fluidly communicate the elastomeric pump 110 and the catheter 170 via the second flow channel 162 in order to dispense the medical fluid to the patient at flow rates and/or pressures less than the third flow condition.

In further operation, as the medical fluid in the elastomeric pump becomes depleted and it is desired to continue administration of the medical fluid to the patient in order to keep the patient's vein open, the body portion 150 of valve member 140 may be advantageously rotated such that the first flow channel 160 may be co-axially aligned with the inlet and outlet apertures 146 and 148 of the base portion 142 and the lumen 167 of the tubing 165. Accordingly, the stem 152 may fluidly communicate the elastomeric pump 110 and the catheter 170 via the first flow channel 160 having the minimal diameter or size. Accordingly, the medical fluid may be dispensed to the patient at flow rates and/or pressures less than the second and third flow conditions and the patient's vein 55 may advantageously be kept open until the clinician is ready to further attend to the patient.

Accordingly, the medical fluid delivery systems of the various embodiments described herein provides various advantages over currently existing medical fluid delivery systems which typically incorporate elastomeric pumps to administer drugs via IV lines.

Currently, manual syringe flushes are administered in order to keep veins open after a catheter has been placed. However, this is an arduous task for clinicians and does not always result in keeping the vein open, as there is not a continuous flushing of the vascular access line. How frequently flushes are administered also depends on the patient; out patients may not get regular flushes resulting in potential occlusions and infections.

The medical fluid delivery systems of the various embodiments described herein advantageously incorporate a keep-vein-open vascular line assist device (referred to herein as a valve member or stop cock valve) capable of keeping a vascular access line open when not in continuous use. The keep-vein-open vascular line assist device of the various embodiments described herein provides continuous flushing of the vascular access line under controlled pressure until the elastomeric pump is emptied. Accordingly, the potential harm to patients associated with catheter occlusions and catheter infections may be reduced or avoided. The keep-vein-open vascular line assist device may also help increase compliance to vascular access maintenance regulations and reduce the amount of time spent by medical professionals manually flushing the line with a syringe. Further advantageously, the keep-vein-open vascular line assist device may also provide a controlled amount of fluid to the patient, especially for those who may be more sensitive to fluid build-up.

The medical fluid delivery systems of the various embodiments described herein incorporate a keep-vein-open vascular line assist device (referred to herein as a valve member or stop cock valve) which advantageously allows a user or medical professional to regulate and/or adjust the flowrate at which the medical fluid may be dispensed even as the natural pressure applied by the elastomeric pump decreases over time. For example, the user or medical professional may initially set the body portion of the valve member to the high pressure/high flow condition through the smallest diameter flow channel, and as the natural pressure applied by the elastomeric pump decreases over time, the user may adjust the flowrate by rotating the body portion of the valve member to a flow channel with a larger diameter in order to compensate for the decrease in natural pressure applied by the pump over time.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 5. The other clauses can be presented in a similar manner.

Clause 1. A system for controlled delivery of a medical fluid, the system comprising: an elastomeric pump containing the medical fluid, the elastomeric pump comprising an elastomeric membrane defining a chamber containing the medical fluid, and a shell surrounding the elastomeric membrane; a valve member fluidly coupled to the elastomeric pump at one side and a catheter at an opposite side for selectively delivering the medical fluid to a vein of a patient, wherein a pressure of the medical fluid is greater than a pressure of the vein by a predetermined value to keep the vein open, and wherein the valve member is movable between an initial closed position, a fully open position where the medical fluid is delivered to the vein of the patient at a predetermined fluid flow rate, and at least one intermediate open flow position where the medical fluid is delivered to the vein of the patient at a flow rate below the predetermined fluid flow rate; and a tubing interposed between and fluidly coupling the elastomeric pump and the valve member.

Clause 2. The system of Clause 1, wherein the valve member comprises a check valve.

Clause 3. The system of Clause 2, wherein the valve member comprises a stopcock valve.

Clause 4. The system of Clause 3, wherein the stopcock valve comprises: a base portion comprising a longitudinally extending body having a lumen extending longitudinally through at least a portion of the base portion; and a body portion comprising a handle and a stem extending longitudinally from the handle and being disposed at least partially in the lumen.

Clause 5. The system of Clause 4, wherein the base portion comprises a cylindrically extending longitudinal body having an outer surface, an inner surface defining the lumen, and a sidewall defined between the inner surface and the outer surface.

Clause 6. The system of Clause 5, wherein the cylindrically extending longitudinal body comprises an inlet aperture extending through the sidewall and an outlet aperture extending through an opposite side of the sidewall of the base portion, the inlet and outlet apertures fluidly coupling the base portion to a lumen of the tubing.

Clause 7. The system of Clause 6, wherein the stem comprises a body including a plurality of flow channels extending laterally though the body.

Clause 8. The system of Clause 7, wherein the plurality of flow channels are longitudinally spaced apart from each other along a circumference of the body.

Clause 9. The system of Clause 8, wherein the plurality of flow channels comprise different sizes.

Clause 10. The system of Clause 9, wherein the plurality of flow channels comprise a first flow channel having a first diameter, a second flow channel having a second diameter, and a third flow channel having a third diameter.

Clause 11. The system of Clause 10, wherein the second diameter is larger than the first diameter and the third diameter is larger than the second diameter.

Clause 12. The system of Clause 11, wherein the inner surface of the base portion comprises a plurality of threads and the stem comprises a plurality of complimentary threads on an outer surface thereof; and wherein, in an engaged configuration of the complimentary threads, the body portion is rotatable about a central axis of the base portion to translate the body portion proximally and distally within the lumen.

Clause 13. The system of Clause 12, wherein the first flow channel is co-axially aligned with the inlet and outlet apertures and the lumen of the tubing in a first flow condition, the second flow channel is co-axially aligned with the inlet and outlet apertures and the lumen of the tubing in a second flow condition, and the third flow channel is co-axially aligned with the inlet and outlet apertures and the lumen of the tubing in a third flow condition.

Clause 14. The system of Clause 13, wherein the first flow condition comprises a first predetermined fluid flow rate, the second flow condition comprises a second predetermined fluid flow rate, and the third flow condition comprises a third predetermined fluid flow rate, and the second predetermined fluid flow rate is greater than the first fluid predetermined flow rate, and the third predetermined fluid flow rate is greater than the second predetermined fluid flow rate.

Clause 15. A system for controlled delivery of a medical fluid, the system comprising: an elastomeric pump containing the medical fluid at a first pressure, the elastomeric pump comprising an elastomeric membrane defining a chamber containing the medical fluid, and a shell surrounding the elastomeric membrane; a valve member comprising (i) a base portion including an inner lumen, an inlet aperture, and an outlet aperture, and (ii) a body portion including a stem disposed at least partially within the inner lumen and including a handle, the stem comprising a plurality of flow channels extending therethrough and defining fluid pathways of different sizes, wherein the valve member fluidly couples the elastomeric pump and a catheter via each of the flow channels for selectively delivering the medical fluid to a patient's vein having a second pressure less than the first pressure by a predetermined value to keep the patient's vein open; and a tubing interposed between and fluidly coupling the elastomeric pump, the valve member, and the catheter.

Clause 16. The system of Clause 15, wherein the valve member is movable between (i) an initial closed position where the stem occludes the inner lumen of the base portion and blocks fluid communication between the elastomeric pump and the catheter, and (ii) a plurality of open positions where each of the flow channels selectively fluidly couples the elastomeric pump and the catheter such that the medical fluid is delivered to the patient's vein at a predetermined fluid flow rate to keep the patient's vein open.

Clause 17. The system of Clause 15, wherein the plurality of flow channels are longitudinally spaced apart from each other along a circumference of the stem.

Clause 18. The system of Clause 15, wherein the plurality of flow channels have different sizes.

Clause 19. The system of Clause 15, wherein an inner surface of the base portion comprises a plurality of threads and the stem comprises a plurality of complimentary threads on an outer surface thereof; and wherein, in an engaged configuration of the complimentary threads, the body portion is rotatable about a central axis of the base portion to translate the body portion proximally and distally within the inner lumen

Clause 20. The system of Clause 15, wherein the plurality of flow channels comprise a first flow channel having a first diameter, a second flow channel having a second diameter, and a third flow channel having a third diameter.

Clause 21. The system of Clause 20, wherein the second diameter is larger than the first diameter and the third diameter is larger than the second diameter.

Clause 22. The system of Clause 20, wherein the first flow channel is co-axially aligned with the inlet and outlet apertures and a lumen of the tubing in a first flow condition, the second flow channel is co-axially aligned with the inlet and outlet apertures and the lumen of the tubing in a second flow condition, and the third flow channel is co-axially aligned with the inlet and outlet apertures and the lumen of the tubing in a third flow condition.

Clause 23. The system of Clause 22, wherein the first flow condition comprises a first predetermined fluid flow rate, the second flow condition comprises a second predetermined fluid flow rate, and the third flow condition comprises a third predetermined fluid flow rate, and the second predetermined fluid flow rate is greater than the first fluid predetermined flow rate, and the third predetermined fluid flow rate is greater than the second predetermined fluid flow rate.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, or operations in the processes or methods disclosed are illustrations of exemplary approaches. Based upon implementation preferences or scenarios, it is understood that the specific order or hierarchy of steps, operations or processes may be rearranged. Some of the steps, operations or processes may be performed simultaneously. In some implementation preferences or scenarios, certain operations may or may not be performed. Some or all of the steps, operations, or processes may be performed automatically, without the intervention of a user. The accompanying method claims present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language of the claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A system for controlled delivery of a medical fluid, the system comprising:
an elastomeric pump containing the medical fluid, the elastomeric pump comprising an elastomeric membrane defining a chamber containing the medical fluid, and a shell surrounding the elastomeric membrane;
a valve member fluidly coupled to the elastomeric pump at one side and a catheter at an opposite side for selectively delivering the medical fluid to a vein of a patient,
wherein a pressure of the medical fluid is greater than a pressure of the vein by a predetermined value to keep the vein open, and
wherein the valve member is movable between an initial closed position, a fully open position where the medical fluid is delivered to the vein of the patient at a predetermined fluid flow rate, and at least one intermediate open flow position where the medical fluid is delivered to the vein of the patient at a flow rate below the predetermined fluid flow rate; and
a tubing interposed between and fluidly coupling the elastomeric pump and the valve member.

2. The system of claim 1, wherein the valve member comprises a check valve.

3. The system of claim 1, wherein the valve member comprises a stopcock valve.

4. The system of claim 3, wherein the stopcock valve comprises:
a base portion comprising a longitudinally extending body having a lumen extending longitudinally through at least a portion of the base portion; and
a body portion comprising a handle and a stem extending longitudinally from the handle and being disposed at least partially in the lumen.

5. The system of claim 4, wherein the base portion comprises a cylindrically extending longitudinal body having an outer surface, an inner surface defining the lumen, and a sidewall defined between the inner surface and the outer surface.

6. The system of claim 5, wherein the cylindrically extending longitudinal body comprises an inlet aperture extending through the sidewall and an outlet aperture extending through an opposite side of the sidewall of the base portion, the inlet and outlet apertures fluidly coupling the base portion to a lumen of the tubing.

7. The system of claim 6, wherein the stem comprises a body including a plurality of flow channels extending laterally though the body.

8. The system of claim 7, wherein the plurality of flow channels are longitudinally spaced apart from each other along a circumference of the body.

9. The system of claim 7, wherein the plurality of flow channels comprise different sizes.

10. The system of claim 7, wherein the plurality of flow channels comprise a first flow channel having a first diameter, a second flow channel having a second diameter, and a third flow channel having a third diameter.

11. The system of claim 7, wherein the inner surface of the base portion comprises a plurality of threads and the stem comprises a plurality of complimentary threads on an outer surface thereof; and
wherein, in an engaged configuration of the complimentary threads, the body portion is rotatable about a central axis of the base portion to translate the body portion proximally and distally within the lumen.

12. The system of claim 7, wherein the first flow channel is co-axially aligned with the inlet and outlet apertures and the lumen of the tubing in a first flow condition, the second flow channel is co-axially aligned with the inlet and outlet apertures and the lumen of the tubing in a second flow condition, and the third flow channel is co-axially aligned with the inlet and outlet apertures and the lumen of the tubing in a third flow condition.

13. A system for controlled delivery of a medical fluid, the system comprising:
an elastomeric pump containing the medical fluid at a first pressure, the elastomeric pump comprising an elastomeric membrane defining a chamber containing the medical fluid, and a shell surrounding the elastomeric membrane;
a valve member comprising (i) a base portion including an inner lumen, an inlet aperture, and an outlet aperture, and (ii) a body portion including a stem disposed at least partially within the inner lumen and including a handle, the stem comprising a plurality of flow channels extending therethrough and defining fluid pathways of different sizes,
wherein the valve member fluidly couples the elastomeric pump and a catheter via each of the flow channels for selectively delivering the medical fluid to a patient's vein having a second pressure less than the first pressure by a predetermined value to keep the patient's vein open; and
a tubing interposed between and fluidly coupling the elastomeric pump, the valve member, and the catheter.

14. The system of claim 13, wherein the valve member is movable between (i) an initial closed position where the stem occludes the inner lumen of the base portion and blocks fluid communication between the elastomeric pump and the catheter, and (ii) a plurality of open positions where each of the flow channels selectively fluidly couples the elastomeric pump and the catheter such that the medical fluid is delivered to the patient's vein at a predetermined fluid flow rate to keep the patient's vein open.

15. The system of claim 14, wherein the plurality of flow channels are longitudinally spaced apart from each other along a circumference of the stem.

16. The system of claim 14, wherein the plurality of flow channels have different sizes.

17. The system of claim 13, wherein an inner surface of the base portion comprises a plurality of threads and the stem comprises a plurality of complimentary threads on an outer surface thereof; and
wherein, in an engaged configuration of the complimentary threads, the body portion is rotatable about a central axis of the base portion to translate the body portion proximally and distally within the inner lumen.

18. The system of claim 14, wherein the plurality of flow channels comprise a first flow channel having a first diameter, a second flow channel having a second diameter, and a third flow channel having a third diameter.

19. The system of claim 18, wherein the first flow channel is co-axially aligned with the inlet and outlet apertures and a lumen of the tubing in a first flow condition, the second flow channel is co-axially aligned with the inlet and outlet apertures and the lumen of the tubing in a second flow condition, and the third flow channel is co-axially aligned with the inlet and outlet apertures and the lumen of the tubing in a third flow condition.

20. The system of claim 19, wherein the first flow condition comprises a first predetermined fluid flow rate, the second flow condition comprises a second predetermined fluid flow rate, and the third flow condition comprises a third predetermined fluid flow rate, and the second predetermined fluid flow rate is greater than the first fluid predetermined flow rate, and the third predetermined fluid flow rate is greater than the second predetermined fluid flow rate.
